(19) United States
(12) Patent Application Publication
(10) Pub. No.: US 2026/0199683 A1
(43) Pub. Date: Jul. 16, 2026

(54) DEEP BRAIN STIMULATION

(71) Applicant: Stardots AB, Uppsala (SE)

(72) Inventors: Daniel Petrini, Uppsala (SE); Lina Uggla, Uppsala (SE); Alexander Medvedev, Täby (SE); Paola Manti, Uppsala (SE)

(21) Appl. No.: 19/448,996

(22) Filed: Jan. 14, 2026

Related U.S. Application Data
(60) Provisional application No. 63/745,978, filed on Jan. 16, 2025.

(51) Int. Cl. A61N 1/36; A61N 1/05; G06T 7/30; G06V 10/40; G06V 10/764; G16H 50/50

(52) U.S. Cl. CPC A61N 1/36139; A61N 1/36132; G06T 7/30; G06V 10/40; G06V 10/764; G16H 50/50; A61N 1/0534; G06T 2207/10081; G06T 2207/10088; G06T 2207/30016; G06V 2201/031

(57) ABSTRACT

A method for supporting deep brain stimulation (DBS) programming includes receiving pre-operative magnetic resonance imaging (MRI) data and post-operative computed tomography (CT) data of a patient having an implanted DBS lead and registers the MRI and CT data into a common spatial reference frame. Anatomical and device-related features, including DBS lead placement and one or more neural regions of interest, are extracted from the registered images. A patient-specific electrical conductivity model of brain tissue is generated based on the MRI data. Using the conductivity model and a representation of the DBS lead, electric field distributions corresponding to one or more DBS contact configurations are simulated. The simulated electric field is optimized by determining stimulation parameters that increase electric field exposure within the neural regions of interest while limiting exposure outside those regions. One or more DBS stimulation settings are output for clinical programming of the DBS lead.

### CROSS REFERENCE TO RELATED APPLICATIONS

This application claims the benefit of U.S. Provisional Patent Application No. 63/745,978, filed Jan. 16, 2025, the contents of which are incorporated herein by reference in its entirety as if fully set forth.

### FIELD

Illustrative embodiments generally relate to medical diagnostics and treatment and, more particularly, various embodiments relate to deep brain stimulation for treatment of patients.

### BACKGROUND

Deep brain stimulation is a treatment in which neural targets are stimulated by electrical pulses delivered through implanted leads to alleviate symptoms in certain neurological and psychiatric disorders. To achieve a beneficial clinical outcome, adjusting the stimulation parameters is a crucial step, which, at present, is a tedious and difficult trial-and-error procedure.

### SUMMARY OF VARIOUS EMBODIMENTS

In accordance with one embodiment, a method for supporting deep brain stimulation (DBS) programming includes receiving, by a computing system, a pre-operative magnetic resonance imaging (MRI) scan and a post-operative computed tomography (CT) scan of a patient having an implanted DBS lead. The MRI scan and the CT scan are registered into a common spatial reference frame, and anatomical and device-related features including at least a spatial placement of the DBS lead and one or more neural regions of interest are extracted from the registered MRI scan and CT scan. A patient-specific electrical conductivity model of brain tissue based on the MRI scan is generated. Using the electrical conductivity model and a representation of the DBS lead, the electric field distribution produced by one or more DBS contact configurations is simulated and consequently optimized. The optimization is delivered by determining at least one stimulation parameter that increases electric field exposure within the one or more neural regions of interest targeted for stimulation while limiting its spread within neural regions outside the one or more neural regions of interest targeted for stimulation. A message is produced including information relating to one or more DBS stimulation settings, such as the optimized stimulation parameter for clinical programming of the DBS lead.

In some embodiments, the one or more neural regions of interest targeted for stimulation or the neural regions outside the one or more neural regions of interest targeted for stimulation include one or more of the following: a subthalamic nucleus (STN) motor subregion, an STN associative subregion, an STN limbic subregion, or a red nucleus.

In some embodiments, generating the patient-specific electrical conductivity model includes classifying voxels of the MRI scan into one or more of gray matter, white matter, or cerebrospinal fluid and assigning tissue-specific electrical conductivity values to the classified voxels.

In some embodiments, simulating the electric field distribution includes numerically solving a quasistatic steady-current equation using a finite element method over a three-dimensional model of brain tissue surrounding the DBS lead.

In some embodiments, simulating the electric field distribution includes simulating monopolar and bipolar DBS contact configurations by applying boundary conditions corresponding to cathodic and anodic contacts on the DBS lead.

In some embodiments, optimizing the simulated electric field distribution includes applying at least two electric field activation thresholds that differ based on whether a spatial location corresponds to gray matter or white matter.

In some embodiments, a first electric field activation threshold is 0.2 V/mm and a second electric field activation threshold is 0.06 V/mm.

In some embodiments, optimizing the simulated electric field distribution includes determining stimulation parameters that include asymmetric current amplitudes distributed across multiple active contacts of the DBS lead.

In some embodiments, the one or more DBS stimulation settings are ranked based on a score that rewards electric field overlap with the one or more neural regions of interest targeted for stimulation, while penalizing the electric field spill outside the one or more neural regions of interest targeted for stimulation.

In some embodiments, the method further includes updating the optimization based on clinical feedback associated with a previously applied DBS stimulation setting.

In accordance with one embodiment, a system for supporting deep brain stimulation (DBS) programming an imaging interface configured to receive a pre-operative magnetic resonance imaging (MRI) scan and a post-operative computed tomography (CT) scan of a patient having an implanted DBS lead. The system includes an image registration pipeline coupled to the imaging interface and configured to spatially register the MRI scan and the CT scan into a common coordinate frame, an anatomical model generator coupled to the image registration pipeline and configured to generate anatomical and device-related models from the registered MRI scan and CT scan, including a spatial placement of the DBS lead and one or more neural regions of interest, and a tissue conductivity model generator configured to generate a patient-specific electrical conductivity model of brain tissue based at least in part on the MRI scan. The system also includes an electric field computation pipeline configured to compute, using the electrical conductivity model and a geometric representation of the DBS lead, an electric field distribution corresponding to one or more DBS contact configurations, a stimulation parameter selector configured to determine at least one stimulation parameter that within the one or more neural regions of interest targeted for stimulation while limiting electric field exposure within neural regions outside the one or more neural regions of interest targeted for stimulation, and a clinical programming interface configured to produce a setting including information relating to one or more DBS stimulation settings including the determined stimulation parameter for clinical programming of the DBS lead.

In some embodiments, the one or more neural regions of interest targeted for stimulation or the neural regions outside the one or more neural regions of interest targeted for stimulation include one or more of: a subthalamic nucleus (STN) motor subregion, an STN associative subregion, an STN limbic subregion, or a red nucleus.

In some embodiments, generating the patient-specific electrical conductivity model includes classifying voxels of the MRI scan into one or more of gray matter, white matter, or cerebrospinal fluid and assigning tissue-specific electrical conductivity values to the classified voxels.

In some embodiments, simulating the electric field distribution includes numerically solving a quasistatic steady-current equation using a finite element method over a three-dimensional model of brain tissue surrounding the DBS lead.

In some embodiments, simulating the electric field distribution includes simulating monopolar and bipolar DBS contact configurations by applying boundary conditions corresponding to cathodic and anodic contacts on the DBS lead.

In some embodiments, optimizing the simulated electric field distribution includes applying at least two electric field activation thresholds that differ based on whether a spatial location corresponds to gray matter or white matter.

In some embodiments, a first electric field activation threshold is 0.2 V/mm and a second electric field activation threshold is 0.06 V/mm.

In some embodiments, optimizing the simulated electric field distribution includes determining stimulation parameters that include asymmetric current amplitudes distributed across multiple active contacts of the DBS lead.

In some embodiments, the one or more DBS stimulation settings are ranked based on a score that rewards electric field overlap with the one or more neural regions of interest targeted for stimulation, while penalizing the electric field spill outside the one or more neural regions of interest targeted for stimulation.

In some embodiments, the instructions further cause the system to update the optimization based on clinical feedback associated with a previously applied DBS stimulation setting.

Illustrative embodiments are implemented as a computer program product having a computer usable medium with computer readable program code thereon. The computer readable code may be read and utilized by a computer system in accordance with conventional processes.

### BRIEF DESCRIPTION OF THE DRAWINGS

Those skilled in the art should more fully appreciate advantages of various embodiments from the following “Description of Illustrative Embodiments,” discussed with reference to the drawings summarized immediately below.

FIG. 1 schematically shows an example deep brain stimulation system in accordance with various embodiments.

FIG. 2 shows an example spatial orientation of leads in accordance with various embodiments.

FIG. 3 shows an example neural structure parcellation in accordance with various embodiments.

FIG. 4 shows an example representation of electrical conductivity maps in accordance with various embodiments.

FIG. 5 shows an example element modeling diagram in accordance with various embodiments.

FIG. 6 shows an example activation diagram in accordance with various embodiments.

FIG. 7 is a table of example results for example simulation configurations in accordance with various embodiments.

FIG. 8 is a flow diagram of an example method of simulating deep brain stimulation in accordance with various embodiments.

FIG. 9 shows an example computing device in accordance with various embodiments.

### DESCRIPTION OF ILLUSTRATIVE EMBODIMENTS

In illustrative embodiments, a method and apparatus performs deep brain stimulation by simulating various configurations and presenting those configuration to a clinician for use to perform the deep brain stimulation. Details of illustrative embodiments are discussed below.

Deep brain stimulation (DBS) is a therapy that treats a variety of neurological and mental disorders by stimulating specific areas of the brain with electric pulses, similar to those that the neural activity is regulated with. At present, DBS is used as a treatment for motor disorders such as Parkinson's disease (PD) and Essential Tremor (ET), but it has also shown to be a promising therapy for psychiatric disorders, such as obsessive-compulsive disorder and depression.

In DBS, a surgeon plans the placement of the electrodes based on pre-operative brain images. Based on the diagnosis of the patient, the DBS leads are implanted in specific target areas of the brain. For PD, the subthalamic nucleus (STN) or the internal Globus Pallidus (GPi) have shown to be beneficial DBS-targets, such that motor symptoms (e.g tremor or stiffness) are alleviated. For PD, DBS does not halt the neurodegenerative process, but it does provide long-term symptomatic relief for the patients.

Although DBS may be an effective treatment, DBS might also induce adverse side effects, such as speech deterioration and affective side effects, for instance depression and apathy. These side effects are often related to electrode displacement and erroneous stimulation settings, leading to a stimulation spill onto regions beyond the target. Finding the optimal stimulation configuration is an act of balance: on one hand, the level of stimulation should be sufficient to obtain the desired treatment response but, on the other hand, exposing the patient to an excessively high stimulation might lead to the aforementioned DBS-induced side effects and fast battery drainage. To prevent such undesirable effects and obtain a beneficial therapeutical outcome, a precise localization of the electrode and carefully chosen stimulation parameters for the device are selected.

After surgery, the clinician tunes the stimulation parameters, (e.g., frequency, pulse-width, and amplitude of current or voltage pulses), according to the patient's response to the treatment. Furthermore, the selection of active lead contacts is performed. Individualized DBS programming is a crucial step, which at present is sometimes conducted through an often time-consuming and costly trial-and-error procedure that is dependent on the experience of the clinician. Therefore, a more sophisticated strategy during the post-surgical stimulation-parameter tuning stage is needed, such that the time and cost spent on the programming procedure is reduced. By introducing a computer-aided parameter-tuning, insights about potential stimulation settings can be gained.

Accordingly, by integrating stimulation modeling tools, clinicians are aided in tuning the stimulation parameters. Based on information about brain anatomy, obtained from pre- and post-operative neuroimages, the stimulation is computationally optimized in terms of maximizing the target stimulation while avoiding neural structures associated with side effects, by using a finite-element model of the brain.

For example, Parkinson's disease is a neurodegenerative disease, in which the cells responsible for production of dopamine in the brain are destroyed. One of the most characteristic symptoms of PD is involuntary shaking, but other symptoms such as stiffness, bradykinesia, and balance problems, occur. Tremor for typically appears in PD at rest.

For patients suffering from PD, DBS is used to address the common movement symptoms. In various embodiments, DBS targets for PD patients are the subthalamaic nucleus (STN) and the globus pallidus internus (GPi), which are both components of the basal ganglia. The side-effects associated with these structures are similar, and include cognitive and mood related effects, as well as speech deterioration.

Based on its structural connectivity, STN stimulation can be divided into three functional territories: the sensorimotor, the limbic, and the associative domain. Sensorimotor stimulation induces motor-related effects, while the ventromedial territories of the STN (limbic and associative) are associated with non-motor side effects. Thus, the former may be sought as a target.

Stimulation of the GPi has also shown to be effective for symptom relief in PD, with a therapeutic outcome comparable to that achieved by STN-DBS. Compared to STN, GPi-stimulation usually involves a lower medication reduction, but has also been demonstrated to be superior in terms of undesirable DBS-induced effects. However, GPi-DBS usually requires higher stimulation amplitudes, and therefore has a shorter battery life compared to STN stimulation. Motor-related adverse effects are believed to be related to stimulation spill into the internal capsule: a bundle of myelinated axons wrapped around the STN and GPi.

Essential Tremor ET is one of the most prevalent movement disorders, characterized by rhythmic shaking of the upper limbs, head, or voice, which usually appear as an action tremor when in motion. Further information relating to DBS modeling tools may be found in “Integration of Deep Brain Stimulation Modeling Tools” (Sundqvist, Lina Wilhelmina), Uppsala University May 2023, which is incorporated herein as if fully set forth.

DBS may also be utilized to address other diseases as well, such as obsessive compulsive disorder (OCD), and other various neurological disorders.

Although further detail is provided below, described herein is a Software as a Medical Device (SaMD) designed to support clinicians in optimizing DBS therapy. It assists in the process of DBS programming, when the stimulation is individualized to the patient, by providing decision support for adjusting DBS settings to maximize symptom relief for patients while minimizing the risk of induced side effects.

In various embodiments, the DBS methods described herein facilitate the DBS programming process by running image-based models to get computer-based predictions of the optimal contact configuration and stimulation amplitude for a specific patient. In various embodiments, software optimizes the electric field to maximally stimulate the target region associated with symptom relief while avoiding forbidden areas that may induce side effects. These models are derived from perioperative medical images routinely used in DBS procedures, specifically pre-operative magnetic resonance imaging (MRI) and post-operative computed tomography (CT) scans.

During DBS programming, the adjustable parameters include the pulse width, pulse frequency and stimulation amplitude, and selection of active electrode contacts. The contacts distributed along the lead's shaft—distributed in a 1-3-3-1, for example, arrange in modern 8-contact directional leads—can be selectively activated or deactivated to fine-tune the spread of stimulation. In various embodiments, a (multi-) monopolar configuration is employed, where one or multiple contacts are set at cathodic (−) polarity, while the implantable pulse generator serve as the anode (+). The frequency and pulse width may be set to fixed values, such as 130 Hz and 60 μs, respectively, while the contacts and amplitude are iteratively adjusted until an adequate DBS setting is found. Conventional techniques of fine tuning the DBS are lengthy, manual, trial-and-error procedures that are dependent on the subjective experience of the clinician.

Again, although further detail is provided below, as described in various embodiments herein, a uniform threshold of 0.2 V/mm may be utilized in various models to estimate the volume of tissue activated. However, this value may not represent the activation of the majority of smaller fibers embedded in subcortical brain structures. To account for the different excitability properties of the neural substrates, one more (second) activation threshold may be introduced equal to, for example, 0.06 V/mm, for each tetrahedron centroid in the mesh. If such centroid lies on a brain structure that carries the gray matter label the second threshold is used, otherwise, if white matter, the 0.2 V/mm is used.

Further, where a monopolar configuration is used, one or more contacts on the lead can be set as cathodes (−) while the IPG is the anode (+), the return path for the current. A bipolar configuration may also be utilized that enables the contacts on the lead to be chosen as anodes and therefore the electric field will be more focused and confined between the chosen anode and cathode. Therefore, to have a broader therapeutic window of optimized setting for the user, a bipolar configuration is introduced in the model as well.

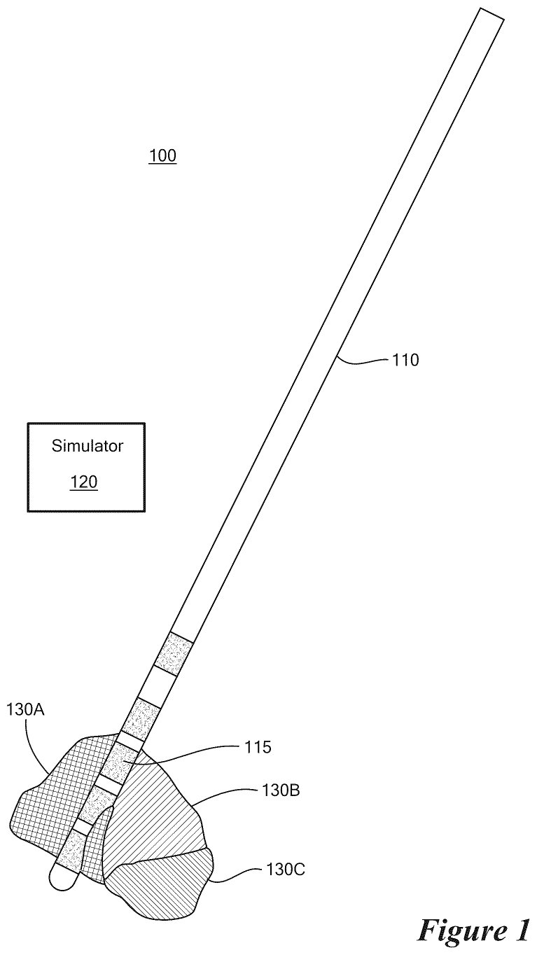

FIG. 1 schematically shows an example DBS system 100 in accordance with various embodiments. As shown in FIG. 1, a probe 110 is inserted into a brain of a patient to provide DBS to the brain. The probe includes one or more electrodes (or leads) 115 which may be energized to generate an electric field within STN regions 130 of the brain proximate to the probe 110. For example, in various embodiments, the STN regions 130 may include subregions STN motor subregion 130A, STN associative subregion 130B, and STN limbic subregion 130C. It should be noted that these subregions are meant as example subregions for STN regions 130 and are non-limiting.

The electrodes 115, in various embodiments, may be energized together or separately to generate an electric field. Additional, combinations of the electrodes 115 may be energized. Accordingly, different neural targets (e.g., subregions 130) may be stimulated by varying the electrical fields generated by energized electrodes 115.

The DBS system 100 also includes a simulator 120, which in various embodiments may include circuitry for performing various functions and method described herein. For example, in various embodiments, the simulator 120 may include simulation circuitry, transmit and receive circuitry, storage circuitry, or the like. Persons of skill in the art will understand that additional circuitries may be resident in the simulator 120. Also, in various embodiments, the simulator 120 may include circuitry and/or components as described below in FIG. 9, for example.

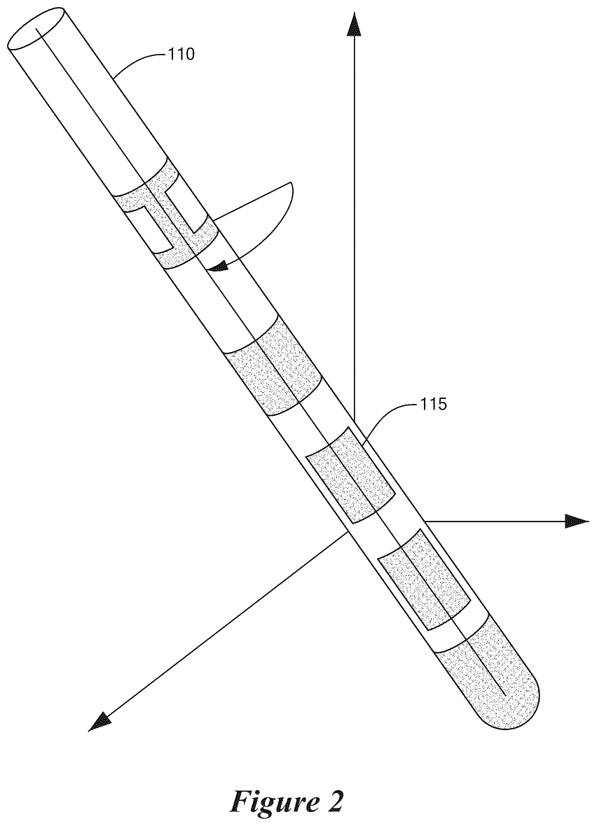

FIG. 2 shows an example spatial orientation of leads in accordance with various embodiments. In various embodiments, the metallic lead leaves artefacts in the post-op CT-scan, which are used to estimate the spatial placement and orientation of the lead.

For example, the post-operative CT scan reveals the lead placement. The artefacts in the image are used to identify the contact row of the electrode implanted in each brain hemisphere. For segmented leads, used to achieve a higher precision of the spread of stimulation, the rotation of the lead shaft is established. A marker on the lead aids in the estimation of the axial rotation. When the placement and orientation of the lead has been established, these parameters can be used to geometrically reconstruct the lead relative to the brain anatomy.

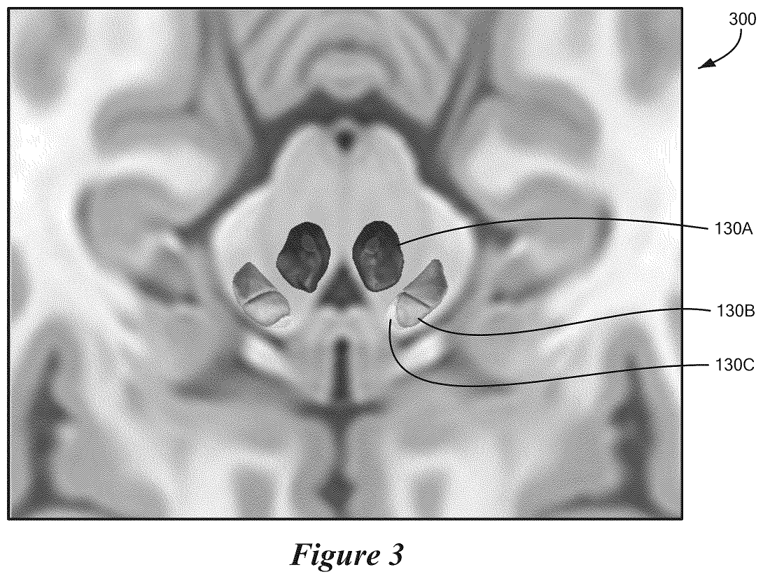

FIG. 3 shows an example neural structure parcellation 300 in accordance with various embodiments. The neural structure parcellation shown in FIG. 3 may include a pre-op MRI that reveals the tissue composition of the brain and is used to identify and locate relevant neural structures (targets and regions to be avoided) using, for example, atlas-based methods. For example, the target regions may include regions 130A, 130B and 130C. However, persons of skill in the art may appreciate other regions may be included.

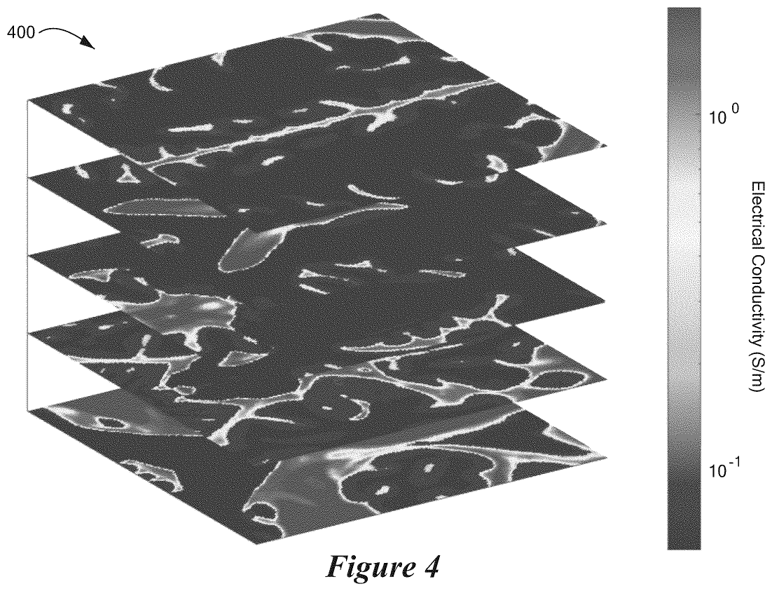

FIG. 4 shows an example representation of electrical conductivity maps 400 in accordance with various embodiments. From the pre-operative MRI, three-dimensional, heterogenous conductivity maps may identify voxels as either cerebrospinal fluid, white matter, or grey matter using K-means segmentation. In various embodiments, linear-interpolation may be used to capture mixture of tissue-intensities at certain voxels.

In various embodiments, the voxels are labelled as white matter, grey matter, and cerebrospinal fluid, with distinct electric conductivities assigned to each tissue type. The linear interpolation then captures voxels with intensities that lie between different tissue types. The result is a heterogeneous conductivity map, an array mapping coordinates to conductivity values.

Being of different modalities and captured at different occasions, the pre-op MR and post-op CT is first be aligned to represent the same reference system. This process is referred to as registration, where the images are aligned into one single coordinate system which is used to mathematically outline the anatomical DBS-setup.

The registered MR provides insights into the patient-specific neural tissue composition and serves two key purposes: segmentation of relevant neural structures and generation of tissue-specific conductivity maps. The CT scan taken after implantation is used to identify the placement and orientation of the lead.

Segmentation of deep neural structures, including the subthalamic nucleus and its subregions—one of the most established targets for DBS in Parkinson's disease—is achieved using, for example, an atlas-based approach as mentioned above, where relevant neural structures are distinguished by referencing a pre-defined image of the standard human brain, in which these anatomical targets are already been identified labelled.

Three-dimensional conductivity maps are constructed through intensity-based segmentation of the tissue composition displayed in the MRI. Using K-means segmentation, the voxels are labelled as white matter, grey matter, and cerebrospinal fluid, with distinct electric conductivities assigned to each tissue type. Linear interpolation is applied to capture voxels with intensities that lie between different tissue types. The result is a heterogeneous conductivity map, an array mapping coordinates to conductivity values

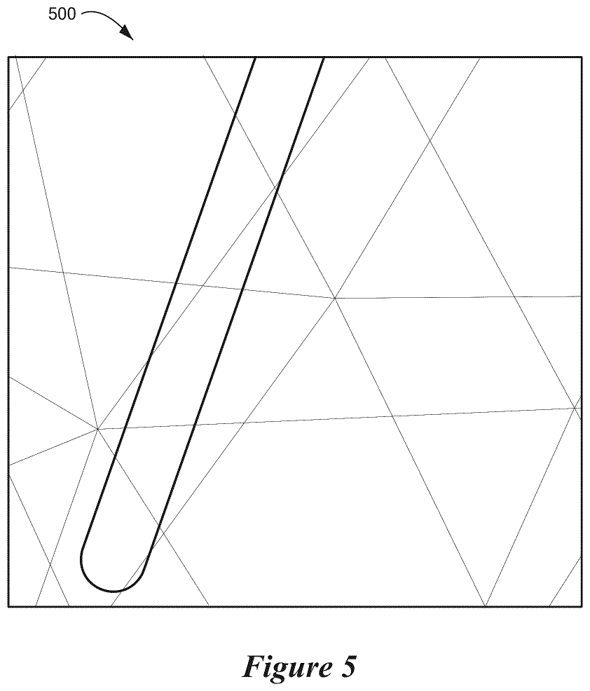

FIG. 5 shows an example element modeling diagram 500 in accordance with various embodiments. In various embodiments, the modeling diagram shows the lead inserted into an area of the brain.

With the localization of the lead, and the three dimensional conductivity maps, the electric field distribution induced by DBS is simulated by numerically solving the equation of steady currents using the finite element method (FEM):

$$∇ \cdot σ ∇ u = 0 ,$$

where σ represents the electrical conductivity (S/mm), which is obtained from the previously established conductivity maps, and u is the electric field potential to for which to solve.

In various embodiments, the boundary conditions of the model are chosen to reflect the stimulation current injected in the tissue through the surface of the contacts of the reconstructed lead shaft. The model is alternately solved for unit stimulation (1 mA) applied to each contact of the DBS lead such as the eight-contact segmented leads of the Boston Scientific Cartesia system. For each configuration, the resulting electric field distribution is calculated. When multiple contacts are active, the overall electric field is obtained by superposing the FEM-solution from the individual contacts.

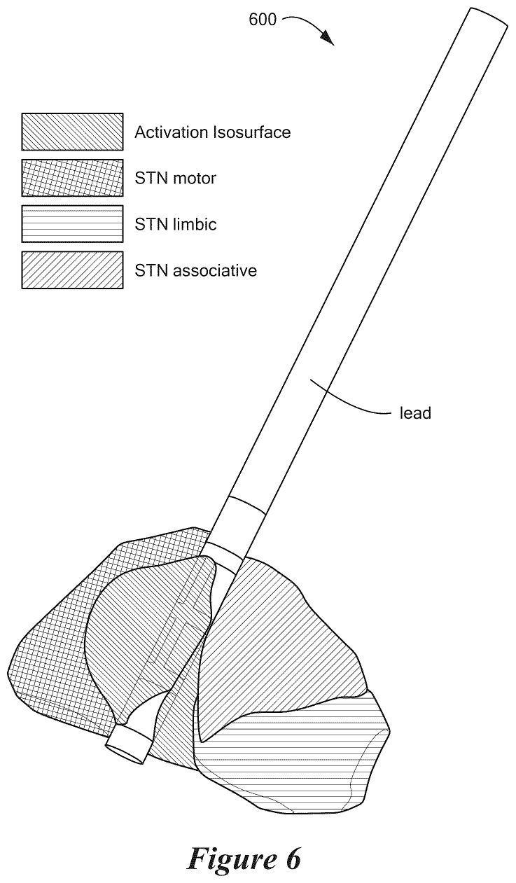

FIG. 6 shows an example activation diagram 600 in accordance with various embodiments. Similar to FIG. 1 above, FIG. 6 shows the STN motor, STN limbic, and STN associate regions, as well as the lead 110. In addition, the simulated activation isosurface is shown depicting the target area that is stimulated by an electric field depending on contacts (e.g., contact configuration) that are energized. As mentioned above, due to the introduction of side effects, the electric field is desired such that a target area to be stimulated is maximized while minimizing activating areas that are not targeted, which in various embodiments, may be referred to as “spill”. Furthermore, stimulation of known forbidden areas associated with side effects is limited/constrained.

That is, the higher the spill, the higher the possibility of introduction of side effects during activation, while the lower the target activation percentage, the lower the activation of areas that may be helpful for treatment. Accordingly, it is beneficial to maximize the target activation area while minimizing the spill. In order to simulate the electric field, various contact configurations may be utilized and energized at different amplitudes or wavelengths, or a combination of both. A more directed field may produce a smaller spill, while a less directed field may produce larger spill.

For example, during an optimization procedure below, three example activation characteristics of the stimulation may be targeted: the rate of target that was activated (target activation), the rate of the forbidden areas that are stimulated (forbidden/constraint activation) and the total amount of stimulation that is not reaching the desired target and is therefore going to waste or stimulating undesired areas—the spill. In various embodiments, the aim is to achieve a high target activation while avoiding stimulation of forbidden neural areas and aiming to keep the spill at low levels.

As shown in FIG. 6, for example, the STN motor subregion may be the target subregion for activation. Accordingly, the electric field induced by the DBS-lead is scaled so that the anatomical targets (e.g. STN motor) are maximally stimulated while the stimulation of forbidden areas associated with side effects (e.g. STN limbic, STN associative) is limited.

However, although the subthalamic nucleus (STN) may be common targets for DBS in Parkinson's Disease, other structures or their subregions may be targeted. For example, modeling may incorporate various neural structures and areas, such as the globus pallidus interna (GPi), ventral intermediate nucleus (ViM), red nucleus, and others. Additionally, the algorithms may consider white matter fiber tracts.

For each contact configuration, a constrained optimization scheme is run and solved as a linear program to find optimal the stimulation current. A certain point is considered as activated when the electric field strength at that point exceeds a predefined threshold. The spatial extent for the activation, the volume of tissue activated (VTA), is estimated for the optimized electric field and the overlap with neural targets is computed.

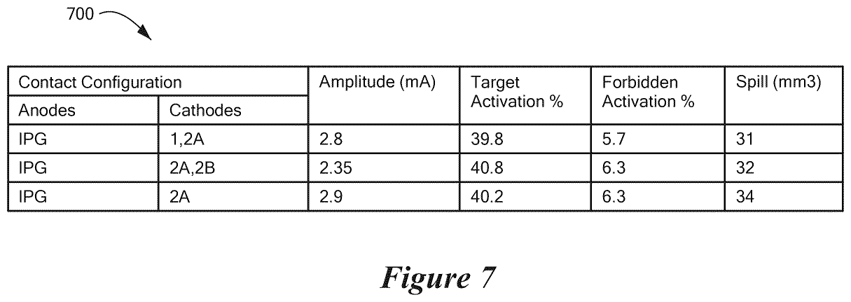

FIG. 7 is a table of example results for example simulation configurations in accordance with various embodiments. As shown in FIG. 7, potential suggested active contacts and amplitudes are shown that provide varying target activation percentages and spill (in mm³). Accordingly, a clinician may utilize these settings based upon a preference for target activation versus spill. As shown in FIG. 7, multiple leads/contacts may be designated as cathodes for simulation and may include designations such as 1, 2A and 2B, etc. In various embodiments, utilizing various cathodes (e.g., 1/2A, 2A/2B, or 2A alone) may provide different simulation outcomes. Depending on the simulation being performed, the designation of a particular contact as 1, 2A, 2B, etc., may vary.

In various embodiments, the optimized settings (e.g., contact configuration and corresponding optimal stimulation amplitude) may be ranked by estimating the overlap between the predicted VTA and the anatomical structure.

This ranking scheme may promote high target activation while penalizing overspill, again which refers to stimulation that occurs outside the intended target area. In various embodiments, a user receives this as a list of the most promising DBS-settings (active contacts and stimulation amplitude). In various embodiments, one or more contacts may receive a first current amplitude, while one or more contacts may receive a second current amplitude.

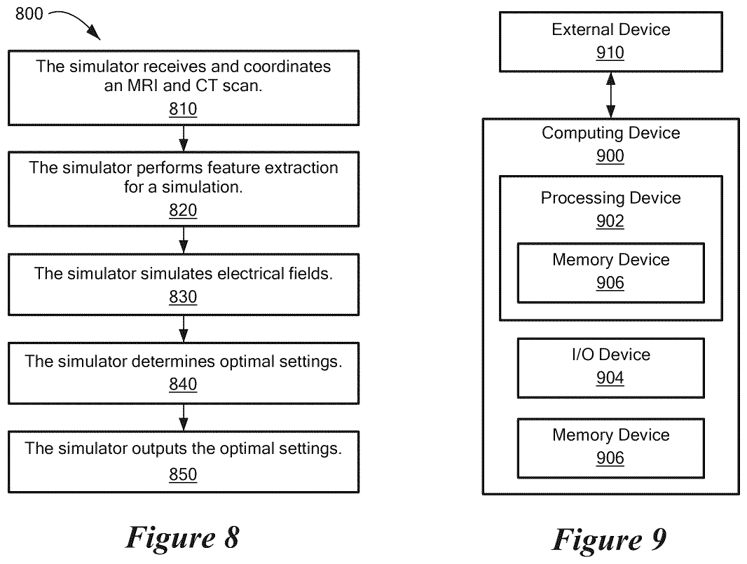

FIG. 8 is a flow diagram of an example method 800 of simulating deep brain stimulation in accordance with various embodiments. In various embodiments, user inputs are utilized, such as:

Perioperative neural images: Pre-op T1-weighted MRI and post-op CT used to create a digital twin.

Lead design: The specific lead model implanted in the patient, selected from a drop-down list.

Stimulation pulse width: If given, this parameter is integrated into the optimization (e.g., default is 60 μs).

Accordingly, the method described herein and above may use the parameters above to produce a model-based prediction on the optimal contact configuration and stimulation amplitude.

At operation 810, the simulator receives and coordinates an MRI and CT scan. For example, the provided medical images are aligned in a process called image-registration so that they represent the same reference system. In this process, the MR is used as the reference onto which the CT is aligned by a geometrical transformation.

With the perioperative images aligned, necessary features are extracted from the registered CT and MRI at operation 820.

In various embodiments, relevant neural structures are identified and localized using, for example, atlas-based methods. A brain atlas is a map of the standard human brain, where relevant anatomical structures are already identified, labelled and outlined. These structures may be reconstructed on a patient-specific level by warping the patient MRI to the standard brain template (MNI average brain), Afterwards, a representation of the regions of interest in the patient specific reference system are obtained using an inverse transform to obtain or by performing simulations in the standard brain space.

As mentioned above, for DBS in Parkinson's disease, one of the most common neural targets is STN, a small lens-shaped structure placed in the basal ganglia. Stimulation of its dorsolateral part is associated with motor control, while spreading to its limbic and associative subregions might lead to psychiatric or cognitive side effects. The internal capsule and (red) nucleus are other subcortical structures near the STN that should be avoided to minimize side effects.

The output from the subcortical parcellation is a point cloud representation of the regions of interest (e.g., the subcortical target area(s) and forbidden regions). As mentioned above, the target areas are areas for which stimulation is to be effected while forbidden regions are regions where spill may occur.

Based on the user-provided MRI data, tissue-specific heterogenous conductivity maps are generated as described above. In a T1-weighted MR image, cerebrospinal fluid appears black, white matter appears white, and grey matter is grey. Three intensity-based clusters are generated using K-means clustering, and the resulting cluster centers correspond to the mid-intensities of the tissue type.

In various embodiments, these mid-intensity points (midpoints) are associated with specific conductivities: 1.71 S/m for cerebrospinal fluid, 0.22 S/m for white matter, and 0.47 S/m for grey matter. Using linear interpolation, with mid-intensities and corresponding conductivities as reference points, a smooth and heterogenous conductivity map is generated for all voxels in the MR.

Once the feature extraction is performed, the simulator simulates the electrical fields at operation 830. In various embodiments, the electrical field simulation is performed by modeling the electrical field.

For example, DBS-induced extracellular electrical field is simulated by employing a volume conductor model to solve the quasistatic equation of steady currents, neglecting capacitive effects that only have a small effect on the induced electrical field:

$$- ∇ \cdot ( σ ( r ) ∇ u ( r ) ) = 0 \qquad ( 1 )$$

where σ(r) is the electrical conductivity of the medium (S/m) and u(r) denotes the electric potential, which is directly related to the electric field, Ē(r), through the relationship Ē(r)=−∇u(r).

Equation (1) is solved separately for each hemisphere separately, as applicable, using the finite element method (FEM).

Using the previously determined lead placement and orientation, the lead geometry is reconstructed for each hemisphere. A predefined, multidomain geometry corresponding to the user-reported lead design is loaded into the model, featuring the lead contacts distributed along the lead shaft, separated by an insulation layer. Using rotational and translational operations, the lead geometry is correctly aligned to reflect the previously derived position and orientation.

As a result of the immune response due to the insertion of a foreign object, an encapsulation layer is formed around the lead. The precise conductivity and thickness of this layer may vary in time and across patients. In an example model, the thickness and conductivity of the encapsulation layer is set to 0.5 mm and 0.18 S/m, respectively.

The brain medium is modelled as a sphere with a radius of 60 mm. In the vicinity of the lead, within 25 mm away from the contact array, the previously derived tissue-specific conductivity values are applied to capture the local heterogeneity. The remaining medium is considered a bulk tissue with a conductivity of 0.1 S/m. To avoid numerical issues arising from high gradient areas, the transition from hetero- to homogenous tissue is gradual.

In the example model, current injection is incorporated by imposing appropriate boundary conditions. In a monopolar configuration, one or multiple lead contacts are set to cathode (−) and the implantable pulse generator, the DBS power-house, is programmed as anode (+).

To simulate an active contact, a Neumann boundary condition is applied, specifying the surface current density J₀ on the face of the stimulating contact ∂Ωᵢ, so that current is injected at this location:

$$\overline{n} \cdot ( σ ∇ u ) = {J}_{0} on \partial {Ω}_{i} \qquad ( 2 )$$

A total injected current of 1 mA, distributed across the face of the active contact, is used for the simulation. Each contact along the lead shaft is sequentially considered as active, while the remaining contacts are kept floating. The exterior of the computational domain is grounded (0 V).

Monopolar simulations are run for each contact on the lead, so that the electrical field distribution induced by a single active contact, while keeping the remining inactive, is obtained. To estimate the electrical field for multiple active monopoles with uniformly distributed current across each active contact, we utilize the linearity of the problem. This allows the estimation of the electrical field contributions from individual poles independently and then fuse them to obtain the solution for unit stimulation with more complex configurations involving multiple active contacts.

In a bipolar configuration, one lead contact is set as the cathode (−) while one contact on the same lead is assigned as the anode (+). In this configuration, only the anode on the lead acts as return path and shapes the field to be confined between the active contacts.

To simulate this configuration, Neumann boundary conditions are applied at both the cathodic and anodic contact surfaces. A surface current density J_{c} is imposed on the cathodic face ∂Ω_{c}:

$$n \cdot ( σ ∇ u ) = {J}_{c} on \partial {Ω}_{c} ,$$

and an equal and opposite current density Jₐ=−J_{c} is applied to the anodic face ∂Ωₐ:

$$n \cdot ( σ ∇ u ) = - {J}_{c} on \partial {Ω}_{a} ,$$

ensuring conservation of current within the domain. The exterior of the computational domain is again set to 0 V using, for example, a Dirichlet boundary condition, providing a distant ground reference but not contributing to the return path in this case.

Simulations may be conducted for all relevant contact pairs to evaluate the localized electric field shaped strictly between the active electrodes.

At operation 840, the simulator determines optimal settings. In various embodiments, the electrical field is optimized. For example, the extracellular electrical field is optimized with respect to the relevant neural structures, such that the stimulation of the target region is maximized while avoiding stimulation of regions associated with side effects, as mentioned above.

A neural response, in this context, is taking place where the electric field strength exceeds a threshold, defined by:

$${E}_{T} = 0.098 \frac{V}{mm} ( 1 + \frac{62 μs}{{T}_{p}} ) \qquad ( 3 )$$

where Tₚ denotes the stimulation pulse width, giving an activation threshold of 0.2 V/mm with the default pulse width of 60 μs. In various embodiments, this threshold is based on the activation of large fibers only and may underrepresent the majority of smaller fibers present in subcortical structures that are impacted differently by the stimulation.

Accordingly, a second threshold may be utilized to better represent the recruitment of these fibers, based on whether readings are performed in the presence of gray matter nuclei or white matter fiber bundles. This information is taken either from the brain structures labels (1=GM, 2=WM) or the conductivity map voxels, so each tetrahedron in the mesh will get a different activation threshold based on whether is centroid is located in gray or white matter.

To consider also the orientation-dependent effect of the stimulation, an alternative activation metric may be integrated (e.g., the AF-3D method), which is unbiased by fiber orientation and may better represent scenarios in which fiber directionality impacts the provided settings.

For a given contact configuration, the stimulation amplitude is optimized with respect to the relevant neural structures, such that the points representing the target Qt (e.g. STN sensorimotor) are maximally stimulated under the constraint that the forbidden region Ω_{f} (e.g. STN limbic, STN associative, red nucleus) remain unstimulated:

$${max}_{α} f ( α ) = α {\sum }_{i ∈ {Ω}_{t}} {E}_{i} \qquad ( 4 )$$

$$s . t . α {E}_{j} < {E}_{T} , for ω of j ∈ {Ω}_{f}$$

Here, α is a scaling factor of the electric field, corresponding to the stimulation amplitude (mA), Eᵢ is the electric field of a point in the target point cloud, and Ej is the electric field strength at a point within the forbidden region. The relaxation factor ω is a parameter, ranging from 0 to 1, indicating the fraction of points within the forbidden region that may excluded from the constraint.

In various embodiments, the problem may also be inverted to minimize the stimulation of points within the forbidden region under the constraint that at least a fraction to of the points in the target exceed the activation threshold:

$${min}_{α} f ( α ) = α {\sum }_{j ∈ {Ω}_{f}} {E}_{j} \qquad ( 5 )$$

$$s . t . α {E}_{i} > {E}_{T} , for ω of i ∈ {Ω}_{t}$$

The linear program is solved for a set of predefined monopolar contact configurations, resulting in a configuration-specific optimal amplitude.

In various embodiments, some DBS systems, enable the user to further fine-tune the stimulation by distributing the stimulation in a non-uniform manner across active contacts—an additional degree of freedom that further advances DBS-programming process. For example, as described above, different contacts may be energized with different amplitudes. Accordingly, contact configurations may be optimized for cases with an asymmetric current distribution over the active contacts. This is achieved by expanding the contact configurations explored in the optimization scheme by defining a set of non-uniform distributions that are considered for every contact configuration.

At operation 850, the simulator outputs the optimal settings. In various embodiments, the optimal settings are utilized by a clinician to energize contacts for DBS.

In some embodiments, a ranking of the settings may be provided. To compare the stimulation effect on anatomical structures for a given configuration and optimized stimulation amplitude, the volume of tissue activated (VTA) and its intersection with relevant subcortical nuclei estimated.

The total amount of stimulated tissue for a specific setting is calculated by first scaling the electrical field strength for a specific contact configuration with the factor a and then estimating the volume encompassed by the points exceeding the activation threshold using the MATLAB alphaShape function. The overlap of the VTA, and the target- and forbidden structures are estimated to produce the following activation metrics for each investigated setting:

$${A}_{t} = \frac{{VTA}_{t}}{{V}_{t}} \qquad ( 6 )$$

$${A}_{f} = \frac{{VTA}_{f}}{{V}_{f}} \qquad ( 7 )$$

$${s}_{R} = 1 - \frac{{VTA}_{t}}{VTA} \qquad ( 8 )$$

where A is a number ranging from 0-1 indicating the activation rate and V corresponds to the total volume. The indices t and f denotes the target and forbidden regions, respectively. The spill rate, sr, estimates the amount of VTA which is outside the target and thereby indicates how well-directed the stimulation field for a specific setting is.

Based on the activation metrics above, the contact configurations along with their respective optimal stimulation amplitude a are ranked according to a score S that rewards a high target activation and penalizes spill and penalizes forbidden activation according to the following:

$$S = {w}_{1} {t}_{A} - {w}_{2} {f}_{A} - {w}_{3} {s}_{R} ,$$

where w₁ to w₃ are weights (>=0) assigned to each factor. In various embodiments, normalized activation metrics may be included to account for the relative variations within the set.

Once the score has been computed for each contact configuration, the user receives a list of the three most promising settings (configuration and stimulation amplitude) for each brain hemisphere.

In various embodiments, model calibration may be performed with clinical effects. For example, a DBS model relying solely on neural imaging data, captured at a fixed point in time, may suffer from the limitation of being static. To capture interactive, real-time aspects to the modelling strategy, clinical effects registered for a specific DBS-setting may be integrated into the optimization scheme.

In various embodiments, clinical effects include both motor response and DBS-induced side effects, such as dysarthria, paraesthesia, muscle contractions, and psychiatric and cognitive effects. Current practice to evaluate motor effects in Parkinson's Disease is the MDS-UPDRS III rating scale, where a score is given for one or more items covering, for instance, rigidity, rest tremor, kinetic tremor, and gait.

Accordingly, these dynamic inputs can be integrated into the modelling component by adjusting the optimization parameters (e.g. by tightening or loosening the relaxation factor ω), based on the clinical output for a specific setting. This approach provides an adaptive and dynamic dimension to the computer-based DBS optimization, while considering per-patient informed settings.

The operations and blocks of any method described above, such as of FIG. 8 are merely illustrative, and variations are contemplated to be within the scope of the present disclosure. In embodiments, the operations and blocks may include other operations not illustrated. In embodiments, the operations and blocks may not include every operation illustrated. In embodiments, the operations and blocks may be implemented in a different order than that illustrated. Such and other embodiments are contemplated to be within the scope of the present disclosure. Persons of skill in the art will appreciate that, although various example components may be described as performing various functions, operations and blocks, other components may perform those functions, operations and blocks described above.

FIG. 9 shows an example computing device in accordance with various embodiments.

For example, FIG. 9 schematically shows a computing device 900 in accordance with various embodiments. The computing device 900 is one example of a computing device which is used to perform one or more operations of process/method 800 illustrated in FIG. 8. The computing device 900 includes a processing device 902, an input/output device 904, and a memory device 906. The computing device 900 may be a stand-alone device, an embedded system, or a plurality of devices configured to perform the functions described with respect to one of the components of system 100.

Furthermore, the computing device 900 may communicate with one or more external devices 910.

The input/output device 904 enables the computing device 900 to communicate with an external device 910. For example, the input/output device 904 may be a network adapter, a network credential, an interface, or a port (e.g., a USB port, serial port, parallel port, an analog port, a digital port, VGA, DVI, HDMI, FireWire, CAT 5, Ethernet, fiber, or any other type of port or interface), among other things. The input/output device 904 may be comprised of hardware, software, or firmware. The input/output device 904 may have more than one of these adapters, credentials, interfaces, or ports, such as a first port for receiving data and a second port for transmitting data, among other things.

The external device 910 may be any type of device that allows data to be input or output from the computing device 900. For example, the external device 910 may be a meter, a control system, a sensor, a mobile device, a reader device, equipment, a handheld computer, a diagnostic tool, a controller, a computer, a server, a printer, a display, a visual indicator, a keyboard, a mouse, or a touch screen display, among other things. Furthermore, the external device 910 may be integrated into the computing device 900. More than one external device may be in communication with the computing device 900. In various embodiments, the external device may be the probe 110.

The processing device 902 may be a programmable type, a dedicated, hardwired state machine, or a combination thereof. The processing device 902 may further include multiple processors, Arithmetic-Logic Units (ALUs), Central Processing Units (CPUs), Digital Signal Processors (DSPs), or Field-programmable Gate Arrays (FPGA), among other things. For forms of the processing device 902 with multiple processing units, distributed, pipelined, or parallel processing may be used. The processing device 902 may be dedicated to performance of just the operations described herein or may be used in one or more additional applications. The processing device 902 may be of a programmable variety that executes processes and processes data in accordance with programming instructions (such as software or firmware) stored in the memory device 906. Alternatively or additionally, programming instructions are at least partially defined by hardwired logic or other hardware. The processing device 902 may be comprised of one or more components of any type suitable to process the signals received from the input/output device 904 or elsewhere, and provide desired output signals. Such components may include digital circuitry, analog circuitry, or a combination thereof.

The memory device 906 in different embodiments may be of one or more types, such as a solid-state variety, electromagnetic variety, optical variety, or a combination of these forms, to name but a few examples. Furthermore, the memory device 906 may be volatile, nonvolatile, transitory, non-transitory or a combination of these types, and some or all of the memory device 906 may be of a portable variety, such as a disk, tape, memory stick, or cartridge, to name but a few examples. In addition, the memory device 906 may store data which is manipulated by the processing device 902, such as data representative of signals received from or sent to the input/output device 904 in addition to or in lieu of storing programming instructions, among other things. As shown in FIG. 3, the memory device 906 may be included with the processing device 902 or coupled to the processing device 902, but need not be included with both.

In various embodiments, the system 100 may include the probe 110 as an external device and the computing device 900.

In various embodiments, the computing device 900 may include an imaging interface configured to receive a pre-operative magnetic resonance imaging (MRI) scan and a post-operative computed tomography (CT) scan of a patient having an implanted DBS lead. The computing device 900 may also include an image registration pipeline coupled to the imaging interface and configured to spatially register the MRI scan and the CT scan into a common coordinate frame.

In various embodiments, the computing device 900 may include an anatomical model generator coupled to the image registration pipeline and configured to generate anatomical and device-related models from the registered MRI scan and CT scan, including a spatial placement of the DBS lead and one or more neural regions of interest.

In various embodiments, the computing device 900 may include a tissue conductivity model generator configured to generate a patient-specific electrical conductivity model of brain tissue based at least in part on the MRI scan.

In various embodiments, the computing device 900 may include an electric field computation pipeline configured to compute, using the electrical conductivity model and a geometric representation of the DBS lead, an electric field distribution corresponding to one or more DBS contact configurations.

In various embodiments, the computing device 900 may include a stimulation parameter selector configured to determine at least one stimulation parameter that increases electric field exposure within the one or more neural regions of interest while limiting electric field exposure outside the one or more neural regions of interest.

In various embodiments, the computing device 900 may include a clinical programming interface configured to produce a setting including information relating to one or more DBS stimulation settings including the determined stimulation parameter for clinical programming of the DBS lead.

The components described above may reside in the processing device 902 of the computing device 900 and executed by the processing device 902. In various embodiments, the components described above may be in the form of circuitry configured to perform the functionality of the components.

Accordingly, methods and systems described above may include an encapsulation layer representing the brain response to a foreign object was already incorporated into the model. However, the electrical conductivity of this layer varies with time, being higher when close to the surgery and decreasing with time. To incorporate the different conductivity of this layer, the date information needed to estimate the state of the patient (either acute or chronic) is retrieved from the patient neuroimages and used to inform the model about the encapsulation conductivity value to use (e.g. 1.8 S/m for the acute stage and 0.125 S/m for the chronic one).

It is contemplated that the various aspects, features, processes, and operations from the various embodiments may be used in any of the other embodiments unless expressly stated to the contrary. Certain operations illustrated may be implemented by a computer executing a computer program product on a non-transient, computer-readable storage medium, where the computer program product includes instructions causing the computer to execute one or more of the operations, or to issue commands to other devices to execute one or more operations.

While the present disclosure has been illustrated and described in detail in the drawings and foregoing description, the same is to be considered as illustrative and not restrictive in character, it being understood that only certain exemplary embodiments have been shown and described, and that all changes and modifications that come within the spirit of the present disclosure are desired to be protected. It should be understood that while the use of words such as “preferable,” “preferably,” “preferred” or “more preferred” utilized in the description above indicate that the feature so described may be more desirable, it nonetheless may not be necessary, and embodiments lacking the same may be contemplated as within the scope of the present disclosure, the scope being defined by the claims that follow. In reading the claims, it is intended that when words such as “a,” “an,” “at least one,” or “at least one portion” are used there is no intention to limit the claim to only one item unless specifically stated to the contrary in the claim. The term “of” may connote an association with, or a connection to, another item, as well as a belonging to, or a connection with, the other item as informed by the context in which it is used. The terms “coupled to,” “coupled with” and the like include indirect connection and coupling, and further include but do not require a direct coupling or connection unless expressly indicated to the contrary. When the language “at least a portion” or “a portion” is used, the item can include a portion or the entire item unless specifically stated to the contrary. Unless stated explicitly to the contrary, the terms “or” and “and/or” in a list of two or more list items may connote an individual list item, or a combination of list items. Unless stated explicitly to the contrary, the transitional term “having” is open-ended terminology, bearing the same meaning as the transitional term “comprising.”

Various embodiments described herein may be implemented at least in part in any conventional computer programming language. For example, some embodiments may be implemented in a procedural programming language (e.g., “C”), or in an object oriented programming language (e.g., “C++”). Other embodiments described herein may be implemented as a pre-configured, stand-alone hardware element and/or as preprogrammed hardware elements (e.g., application specific integrated circuits, FPGAs, and digital signal processors), or other related components.

In an alternative embodiment, the disclosed apparatus and methods (e.g., see the various flow charts described above) may be implemented as a computer program product for use with a computer system. Such implementation may include a series of computer instructions fixed either on a tangible, non-transitory medium, such as a computer readable medium (e.g., a diskette, CD-ROM, ROM, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World Wide Web). In fact, some embodiments may be implemented in a software-as-a-service model (“SAAS”) or cloud computing model. Of course, some embodiments described herein may be implemented as a combination of both software (e.g., a computer program product) and hardware. Still other embodiments described herein are implemented as entirely hardware, or entirely software.

The embodiments described herein described above are intended to be merely exemplary; numerous variations and modifications will be apparent to those skilled in the art. Such variations and modifications are intended to be within the scope of the present application as defined by any of the appended claims. It shall nevertheless be understood that no limitation of the scope of the present disclosure is hereby created, and that the present disclosure includes and protects such alterations, modifications, and further applications of the exemplary embodiments as would occur to one skilled in the art with the benefit of the present disclosure.

1. A method for supporting deep brain stimulation (DBS) programming, comprising:
receiving, by a computing system, a pre-operative magnetic resonance imaging (MRI) scan and a post-operative computed tomography (CT) scan of a patient having an implanted DBS lead;
registering the MRI scan and the CT scan into a common spatial reference frame;
extracting, from the registered MRI scan and CT scan, anatomical and device-related features including at least a spatial placement of the DBS lead and one or more neural regions of interest targeted for stimulation;
generating a patient-specific electrical conductivity model of brain tissue based on the MRI scan;
simulating, using the electrical conductivity model and a representation of the DBS lead, an electric field distribution produced by one or more DBS contact configurations;
optimizing the simulated electric field distribution by determining at least one stimulation parameter that increases electric field exposure within the one or more neural regions of interest targeted for stimulation while limiting exposure within neural regions outside the one or more neural regions of interest targeted for stimulation; and
producing a setting message comprising information relating to one or more DBS stimulation settings including the determined stimulation parameter for clinical programming of the DBS lead.

2. The method of claim 1, wherein the one or more neural regions of interest targeted for stimulation or the neural regions and outside the one or more neural regions of interest targeted for stimulation include one or more of the following: a subthalamic nucleus (STN) motor subregion, an STN associative subregion, an STN limbic subregion, or a red nucleus.

3. The method of claim 1, wherein generating the patient-specific electrical conductivity model comprises classifying voxels of the MRI scan into one or more of gray matter, white matter, or cerebrospinal fluid and assigning tissue-specific electrical conductivity values to the classified voxels.

4. The method of claim 1, wherein simulating the electric field distribution comprises numerically solving a quasistatic steady-current equation using a finite element method over a three-dimensional model of brain tissue surrounding the DBS lead.

5. The method of claim 1, wherein simulating the electric field distribution includes simulating monopolar and bipolar DBS contact configurations by applying boundary conditions corresponding to cathodic and anodic contacts on the DBS lead.

6. The method of claim 1, wherein optimizing the simulated electric field distribution comprises applying at least two electric field activation thresholds that differ based on whether a spatial location corresponds to gray matter or white matter.

7. The method of claim 6, wherein a first electric field activation threshold is 0.2 V/mm and a second electric field activation threshold is 0.06 V/mm.

8. The method of claim 1, wherein optimizing the simulated electric field distribution comprises determining stimulation parameters that include asymmetric current amplitudes distributed across multiple active contacts of the DBS lead.

9. The method of claim 1, wherein the one or more DBS stimulation settings are ranked based on a score that rewards electric field overlap with the one or more neural regions of interest targeted for stimulation and penalizes electric field spill outside the one or more neural regions of interest targeted for stimulation.

10. The method of claim 1, further comprising updating the optimization based on clinical feedback associated with a previously applied DBS stimulation setting.

11. A system for supporting deep brain stimulation (DBS) programming, comprising:
an imaging interface configured to receive a pre-operative magnetic resonance imaging (MRI) scan and a post-operative computed tomography (CT) scan of a patient having an implanted DBS lead;
an image registration pipeline coupled to the imaging interface and configured to spatially register the MRI scan and the CT scan into a common coordinate frame;
an anatomical model generator coupled to the image registration pipeline and configured to generate anatomical and device-related models from the registered MRI scan and CT scan, including a spatial placement of the DBS lead and one or more neural regions of interest targeted for stimulation;
a tissue conductivity model generator configured to generate a patient-specific electrical conductivity model of brain tissue based at least in part on the MRI scan;
an electric field computation pipeline configured to compute, using the electrical conductivity model and a geometric representation of the DBS lead, an electric field distribution corresponding to one or more DBS contact configurations;
a stimulation parameter selector configured to determine at least one stimulation parameter that increases electric field exposure within the one or more neural regions of interest targeted for stimulation while limiting electric field exposure within neural regions outside the one or more neural regions of interest targeted for stimulation; and
a clinical programming interface configured to produce a setting including information relating to one or more DBS stimulation settings including the determined stimulation parameter for clinical programming of the DBS lead.

12. The system of claim 11, wherein the one or more neural regions of interest targeted for stimulation or the neural regions outside the one or more neural regions of interest targeted for stimulation include one or more of: a subthalamic nucleus (STN) motor subregion, an STN associative subregion, an STN limbic subregion, or a red nucleus.

13. The system of claim 11, wherein generating the patient-specific electrical conductivity model comprises classifying voxels of the MRI scan into one or more of gray matter, white matter, or cerebrospinal fluid and assigning tissue-specific electrical conductivity values to the classified voxels.

14. The system of claim 11, wherein simulating the electric field distribution comprises numerically solving a quasistatic steady-current equation using a finite element method over a three-dimensional model of brain tissue surrounding the DBS lead.

15. The system of claim 11, wherein simulating the electric field distribution includes simulating monopolar and bipolar DBS contact configurations by applying boundary conditions corresponding to cathodic and anodic contacts on the DBS lead.

16. The system of claim 11, wherein optimizing the simulated electric field distribution comprises applying at least two electric field activation thresholds that differ based on whether a spatial location corresponds to gray matter or white matter.

17. The system of claim 16, wherein a first electric field activation threshold is 0.2 V/mm and a second electric field activation threshold is 0.06 V/mm.

18. The system of claim 11, wherein optimizing the simulated electric field distribution comprises determining stimulation parameters that include asymmetric current amplitudes distributed across multiple active contacts of the DBS lead.

19. The system of claim 11, wherein the one or more DBS stimulation settings are ranked based on a score that rewards electric field overlap with the one or more neural regions of interest targeted for stimulation and penalizes electric field spill outside the one or more neural regions of interest targeted for stimulation.

20. The system of claim 11, wherein the instructions further cause the system to update the optimization based on clinical feedback associated with a previously applied DBS stimulation setting.

21. A computer program product for use on a computer system, the computer program product comprising a tangible, non-transient computer usable medium having computer readable program code thereon, the computer readable program code comprising:
program code for receiving, by a computing system, a pre-operative magnetic resonance imaging (MRI) scan and a post-operative computed tomography (CT) scan of a patient having an implanted DBS lead;
program code for registering the MRI scan and the CT scan into a common spatial reference frame;
program code for extracting, from the registered MRI scan and CT scan, anatomical and device-related features including at least a spatial placement of the DBS lead and one or more neural regions of interest targeted for stimulation;
program code for generating a patient-specific electrical conductivity model of brain tissue based on the MRI scan;
program code for simulating, using the electrical conductivity model and a representation of the DBS lead, an electric field distribution produced by one or more DBS contact configurations;
program code for optimizing the simulated electric field distribution by determining at least one stimulation parameter that increases electric field exposure within the one or more neural regions of interest targeted for stimulation while limiting electric field exposure within neural regions outside the one or more neural regions of interest targeted for stimulation; and
program code for producing a setting message comprising information relating to one or more DBS stimulation settings including the determined stimulation parameter for clinical programming of the DBS lead.
